(19) United States
(12) Patent Application Publication
(10) Pub. No.: US 2008/0195062 A1
(43) Pub. Date: Aug. 14, 2008

(54) SAMPLING OF BLOOD ANALYTES

(75) Inventor: RICHARD CAPRIOLI, Brentwood, TN (US)

Correspondence Address: FULBRIGHT & JAWORSKI L.L.P., 600 CONGRESS AVE., SUITE 2400, AUSTIN, TX, 78701, US

(73) Assignee: Vanderbilt University

(21) Appl. No.: 11/422,748

(22) Filed: Jun. 7, 2006

Related U.S. Application Data
(60) Provisional application No. 60/688,618, filed on Jun. 8, 2005.
(60) Provisional application No. 60/711,019, filed on Aug. 24, 2005.

(51) Int. Cl. A61M 5/178

(52) U.S. Cl. 604/264

(57) ABSTRACT

The present invention provides for devices and methods of sampling and analyzing body fluid analytes, including proteins, nucleic acids, drugs, carbohydrates and lipids. Body fluids include blood, serum, sputum, saliva, urine, cerebrospinal fluid, and ascites.

This application claims benefit of priority to U.S. Provisional Application with Ser. Nos. 60/688,618, filed Jun. 8, 2005, and 60/711,019, filed Aug. 24, 2005, the entire contents of each of these applications being hereby incorporated by reference in their entirety.

### BACKGROUND OF THE INVENTION

1. Field of the Invention

The present invention relates generally to the fields of hematology and diagnostic medicine. More particularly, it concerns the analysis of biological fluid analytes using a device inserted into a fluid source, particularly useful for sampling of less abundant or rare analytes.

2. Description of Related Art

Diagnosis or monitoring of a disease process or disorder often requires or can be aided by analysis of a subject's blood and may specifically involve the analysis of a particular component or components found in the blood. Serum or plasma biomarkers hold enormous promise for diagnosing, evaluating prognosis and even treating patients.

Methods have been discussed for the collection of blood or serum and the subsequent enrichment or purification of blood components for use in these diagnostic techniques. For example, Nedelkov et al (2001) describe the use of surface-immobilized ligands to allow enrichment of a binding protein of interest prior to analysis of the protein of interest via mass spectrometry. This method may be applied to analysis of components of plasma collected from human subjects (Nedelkov et al., 2003). Kiernan et al. also describe methods for affinity capture of specified proteins present in a sample as a purification step which may be performed prior to mass spectrometry analysis (Kiernan et al., 2002a; 2002b). Kern et al. (2004) describe a method in which sera was collected from donors and then spotted onto a protein chip that displayed polyclonal antibodies on its surface. The chip was incubated to allow binding between the serum antigens of interest and the antibodies displayed on the chip. Following a series of washing steps, the antigens on the chip were analyzed via mass spectrometry.

The methods described above utilize affinity purification or enrichment of blood components to aid in the analysis of components of interest. These methods perform the purification or enrichment step following collection of the sample and prior to analysis of the components of interest. A major problem associated with such methods is that relatively small amounts of diagnostic analytes are present at any given time and are diluted in liters of blood, serum, urine, CSF, etc. Assaying such components is difficult due to the exceedingly small concentration of the component or components of interest and the presence of numerous other components in collected samples. Thus, there remains a need for enrichment and purification of a analytes during collection of liquid samples, thus overcoming a deficiency in the previously described methods.

### SUMMARY OF THE INVENTION

Thus, in accordance with the present invention, this is provided a method for the analysis of analytes in biological fluids such as blood, serum, saliva, sputum, urine, cerebrospinal fluid, and ascites, using a device inserted into a patient such that the device is in contact with the fluid. In some embodiments, this device comprises a housing suitable for disposition in a subject, a probe comprising a support coupled to the housing, and a binding agent coupled to the support. The binding agent will to one or more analytes in the fluid of the subject. In certain embodiments, the housing of the invention is a hypodermic needle or syringe. In other embodiments, the housing of the invention is a catheter. The probe of the device may comprise a support coupled to the housing. In individual embodiments, the support may be a stent, a filter, a chip or a membrane, or the support may be comprised of a polymer, a resin, glass, or a porous material. In certain embodiments, the probe may comprise only a support and binding agent, whereas in other embodiments, the probe may comprise at least a support and binding agent. In specific embodiments, the probe may be positioned inside or outside of the housing. The binding agent may be located on or in the support and may exhibit affinity for a component or components present in a fluid. In a specific embodiment, the binding agent may be a peptide. In a further embodiment, the binding agent may be a polypeptide such as an antibody or receptor. In other embodiments, the binding agent may be a drug, a nucleic acid, a lipid, a glycolipid, a carbohydrate, a toxin, an antigen, a hapten or an enzyme substrate.

In certain aspects of the invention, the device comprises only the probe and binding agent. In these aspects of the invention, the probe may comprise only a support and binding agent, or the probe may comprise at least a support and binding agent.

In some embodiments of the invention, the device allows recovery of an analyte from the fluid of a subject based on a binding affinity of the analyte for the support of the invention. As another aspect of the invention, the recovered analyte may be subsequently detected and/or analyzed. In some embodiments, detection or analysis of the recovered analyte follows further separation of the analyte from other molecules bound to the support.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

The terms “comprise” (and any form of comprise, such as “comprises” and “comprising”), “have” (and any form of have, such as “has” and “having”), “contain” (and any form of contain, such as “contains” and “containing”), and “include” (and any form of include, such as “includes” and “including”) are open-ended linking verbs. As a result, a device or a method that “comprises,” “has,” “contains,” or “includes” one or more elements possesses those one or more elements, but is not limited to possessing only those one or more elements or steps. Likewise, an element of a device or method that “comprises,” “has,” “contains,” or “includes” one or more features possesses those one or more features, but is not limited to possessing only those one or more features. Furthermore, an agent (e.g., a binding agent) that is coupled to a structure (e.g., a probe) with a material (e.g., a support) is coupled to the structure using at least the recited material.

### BRIEF DESCRIPTION OF THE DRAWINGS

The following drawings form part of the present specification and are included to further demonstrate certain aspects of the present invention. The invention may be better understood by reference to the drawings in combination with the detailed description of illustrative embodiments presented herein. The following drawings illustrate by way of example and not by limitation.

FIG. 1 shows one embodiment of the present invention that includes a housing, a probe and a binding agent.

FIG. 2 shows another embodiment of the present invention that includes a housing, a probe, a support and a binding agent.

### DESCRIPTION OF ILLUSTRATIVE EMBODIMENTS

### I. Device Structure

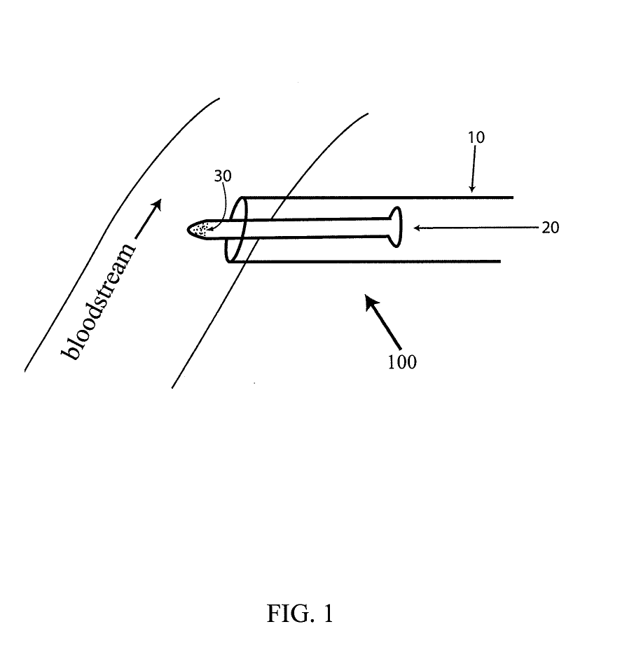

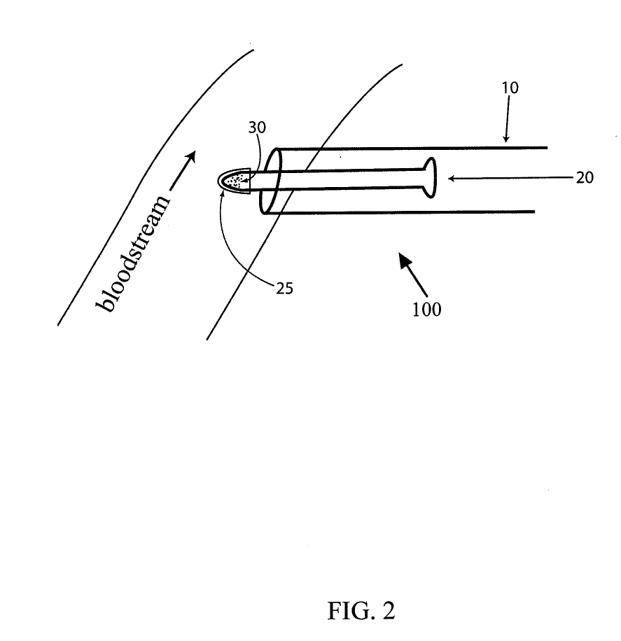

Some embodiments of the present devices, such as device 100 shown in FIG. 1, comprise a housing 10, a probe 20, and a binding agent 30. Other embodiments of the present devices, such as device 100 shown in FIG. 2, comprise a housing 10, a probe 20, and a binding agent 30 coupled to the probe 20 with at least a support 25. Still further embodiments of the present devices do not include housing 10, and do include probe 20 and binding agent 30 or do include probe 20, binding agent 30 and support 25.

A. Housing

A housing according to the invention may comprise any material which is suitable for insertion into a subject. Examples of materials which are suitable for insertion into a subject include biocompatible materials which are nontoxic and sterilizable. In particular embodiments, the housing may be a hypodermic needle, a syringe or a catheter.

In other embodiments, the housing may be optimized for specific applications by coating the housing with an anticoagulant. An anticoagulant is defined as any substance that inhibits blood clot formation. Examples of anticoagulants include, but are not limited to, bishydroxycoumarin (Dicumarol), warfarin, and heparin. In further embodiments, the housing may be optimized for a specific application by coating the housing with a blocking agent. Blocking agents may be employed to inhibit or block a biological process. Examples of blocking agents include, but are not limited to, alpha-adrenergic blockers, beta-adrenergic blockers, antihypertensive drugs, angiotensin blockers, angiotensin receptor blockers, TNF-alpha blockers, ion channel blockers, neuromuscular blocking agents, and thyroid blocking agents. Blocking agents may also be employed to inhibit interactions between a blood component or components and the housing of the device.

B. Probe

A probe according to the invention may comprise a support that is coupled to the housing. In particular embodiments, the probe may be positioned by some means inside or outside of the housing. For example, the probe may be positioned completely inside the housing or may be positioned so that the probe extends beyond the housing in order to allow increased interaction between the fluid and the support. In further embodiments, a cable, plunger or piston may be employed to correctly position the probe with respect to the housing.

A probe according to the invention may comprise only a binding agent located on or in a support as depicted, for example, in FIG. 1. In other aspects of the invention, the probe may comprise at least a binding agent located on or in a support. In certain embodiments of the invention, the support may serve to couple the binding agent to the probe as depicted, for example, in FIG. 2. In specific embodiments, the support may comprise a stent, a filter, a chip or a membrane. In further embodiments, the support may be comprised of a polymer, a resin or glass. In yet another embodiment, the support may be porous so as to allow increased surface area for bloodstream components to contact the binding agent. It is to be understood that support 25 is depicted generically in FIG. 2, and, as will be clear to those of ordinary skill in the art from the examples of suitable supports described in this disclosure, may take many forms that differ in practice from the generic depiction.

In further embodiments, the support may be optimized for a specific application by coating the support with an anticoagulant or blocking agent. Examples and uses of such anticoagulants and blocking agents include, but are not limited to, those listed in the preceding section.

C. Binding Agent

The device of the invention may comprise a binding agent coupled to the support. This binding agent may exhibit affinity for one or more components found in fluids. Binding agents include any agent which can associate with analytes of interest. Examples of binding agents include, but are not limited to, ligands, peptides, polypeptides, fusion proteins, antibodies, antibody fragments, receptors, drugs, nucleic acids, lipids, glycolipids, carbohydrates, toxins, antigens, haptens, diagnostic agents, small molecules, chemicals or enzyme substrates.

### II. Use of the Device

It is contemplated that the device of the invention may be inserted into a subject, for example, arterial or venous bloodstream, cerebrospinal cavity, bladder or urinary tract, mouth, trachea or esophagus. In some embodiments, the device may remain in the subject for any duration of time between about one minute and about 24 hours. In specific embodiments, the device may remain in the subject for about two minutes, about an hour, or about six hours.

In certain embodiments, binding of an analyte or analytes to the probe may result in a detectable event. Examples of such a detectable event include, but are not limited to, a color change, generation of a fluorescent signal, a chemical reaction or generation of an electrical signal.

In a specific embodiment, the invention can be used to recover an analyte by inserting the device into the subject such that the support is brought into contact with the subject's fluid, and the device and/or support is subsequently removed from the subject.

In yet another embodiment of the invention, the device may also deliver an exogenous agent to the subject. For example, the device may deliver a diagnostic agent to the subject, and the diagnostic agent subsequently binds to the support, perhaps following a putative modification which occurs inside the subject.

In other embodiments, analytes of interest may be further purified away from other molecules bound to the support by removing the analytes from the support. In yet other aspects of the invention, the analytes of interest may be further purified after removal from the support. Examples of methods useful in purification of analytes include, but are not limited to, centrifugation, filtration, enzyme digestion or immunologic separation as well as separation of components by size, charge or affinity. Such purification or separation methods are well known in the art and include, but are not limited to, separation by chromatography, electrophoresis or isoelectric point. Additionally, U.S. Patent Application No. 20020098595 describes two-phase protein separation techniques and is incorporated herein by reference. In still further embodiments, analytes of interest may be further purified away from other molecules bound to the support by removing these other molecules from the support. For example, washing steps may be employed to remove other molecules while maintaining the binding interaction between the support and the analytes of interest.

### III. Detection/Analysis of Analytes

Recovered analytes may be detected and/or analyzed. For example, one of skill in the art may choose to employ embodiments of the invention to collect from a subject a sample enriched for a clinically relevant biomarker. Detection and/or analysis of such a biomarker in terms of its presence, quantity or characteristics may be indicative of a diagnosis, prognosis or potential treatment option. Methods of detection and/or analysis include, but are not limited to, those listed below.

A. Immunodetection of Peptides, Polypeptides or Proteins

In accordance with the present invention, methods are provided for detecting peptides, polypeptides or proteins. One such approach is to perform protein identification with the use of antibodies. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. The term “antibody” also refers to any antibody-like molecule that has an antigen-binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies, both polyclonal and monoclonal, are also well known in the art (See, e.g., Antibodies: A Laboratory Manual, Cold Spring Harbor Laboratory, 1988; incorporated herein by reference).

In accordance with the present invention, immunodetection methods are provided. Some immunodetection methods include enzyme-linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle & Ben-Zeev O, 1999; Gulbis & Galand, 1993; De Jager et al., 1993; and Nakamura et al., 1987, each incorporated herein by reference.

In general, the immunobinding methods include obtaining a sample suspected of containing a relevant peptide or polypeptide, and contacting the sample with a first antibody under conditions effective to allow the formation of immunocomplexes. In terms of antigen detection, the biological fluid sample analyzed may be any sample that is suspected of containing an antigen, such as, for example, a fluid sample obtained by using the devices and/or methodologies presented herein.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first step biotinylated, monoclonal or polyclonal antibody is used to detect the target antigen(s), and a second step antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

As detailed above, immunoassays are in essence binding assays. Certain immunoassays are the various types of enzyme-linked immunosorbent assays (ELISAs) and radioimmunoassays (RIA) known in the art. However, it will be readily appreciated that detection is not limited to such techniques, and Western blotting, dot blotting, FACS analyses, and the like may also be used.

In one exemplary ELISA, the antibodies are immobilized onto a selected surface that exhibits affinity for a peptide, polypeptide or protein, such as a well in a polystyrene microtiter plate. Then, a test composition suspected of containing the antigen, such as a clinical sample, is added to the wells. After binding and washing to remove non-specifically bound immune complexes, the bound antigen may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

Another ELISA in which the antigens are immobilized, involves the use of antibody competition in the detection. In this ELISA, labeled antibodies against an antigen are added to the wells, allowed to bind, and detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background. The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

B. Mass Spectrometry

By exploiting the intrinsic properties of mass and charge, mass spectrometry (MS) can resolve and confidently identify a wide variety of complex compounds, including proteins. Traditional quantitative MS has used electrospray ionization (ESI) followed by tandem MS (MS/MS) (Chen et al., 2001; Zhong et al., 2001; Wu et al., 2000) while newer quantitative methods are being developed using matrix assisted laser desorption/ionization (MALDI) followed by time of flight (TOF) MS (Bucknall et al., 2002; Mirgorodskaya et al., 2000; Gobom et al., 2000). In accordance with the present invention, one can generate mass spectrometry profiles that are useful for detecting, identifying, and/or analyzing blood components. It is also contemplated in certain aspects of the invention that the device may comprise or incorporate a probe that is suitable for direct insertion into a mass spectrometer and subsequent analysis of components of interest.

1. ESI

ESI is a convenient ionization technique developed by Fenn and colleagues (Fenn et al., 1989) that is used to produce gaseous ions from highly polar, mostly nonvolatile biomolecules, including lipids. The sample is injected as a liquid at low flow rates (1-10 μL/min) through a capillary tube to which a strong electric field is applied. The field generates additional charges to the liquid at the end of the capillary and produces a fine spray of highly charged droplets that are electrostatically attracted to the mass spectrometer inlet. The evaporation of the solvent from the surface of a droplet as it travels through the desolvation chamber increases its charge density substantially. When this increase exceeds the Rayleigh stability limit, ions are ejected and ready for MS analysis.

A typical conventional ESI source consists of a metal capillary of typically 0.1-0.3 mm in diameter, with a tip held approximately 0.5 to 5 cm (but more usually 1 to 3 cm) away from an electrically grounded circular interface having at its center the sampling orifice, such as described by Kabarle et al. (1993). A potential difference of between 1 to 5 kV (but more typically 2 to 3 kV) is applied to the capillary by power supply to generate a high electrostatic field (106 to 10⁷ V/m) at the capillary tip. A sample liquid carrying the analyte to be analyzed by the mass spectrometer, is delivered to the tip through an internal passage from a suitable source (such as from a chromatograph or directly from a sample solution via a liquid flow controller). By applying pressure to the sample in the capillary, the liquid leaves the capillary tip as small highly electrically charged droplets and further undergoes desolvation and breakdown to form single or multicharged gas phase ions in the form of an ion beam. The ions are then collected by the grounded (or negatively charged) interface plate and led through an the orifice into an analyzer of the mass spectrometer. During this operation, the voltage applied to the capillary is held constant. Aspects of construction of ESI sources are described, for example, in U.S. Pat. Nos. 5,838,002; 5,788,166; 5,757,994; RE 35,413; and 5,986,258.

2. ESI/MS/MS

In ESI tandem mass spectroscopy (ESI/MS/MS), one is able to simultaneously analyze both precursor ions and product ions, thereby monitoring a single precursor product reaction and producing (through selective reaction monitoring (SRM)) a signal only when the desired precursor ion is present. When the internal standard is a stable isotope-labeled version of the analyte, this is known as quantification by the stable isotope dilution method. This approach has been used to accurately measure pharmaceuticals (Zweigenbaum et al., 2000; Zweigenbaum et al., 1999) and bioactive peptides (Desiderio et al., 1996; Lovelace et al., 1991). Newer methods are performed on widely available MALDI-TOF instruments, which can resolve a wider mass range and have been used to quantify metabolites, peptides, and proteins. Larger molecules such as peptides can be quantified using unlabeled homologous peptides as long as their chemistry is similar to the analyte peptide (Duncan et al., 1993; Bucknall et al., 2002). Protein quantification has been achieved by quantifying tryptic peptides (Mirgorodskaya et al., 2000). Complex mixtures such as crude extracts can be analyzed, but in some instances, sample clean up is required (Nelson et al., 1994; Gobom et al., 2000).

3. SIMS

Secondary ion mass spectroscopy, or SIMS, is an analytical method that uses ionized particles emitted from a surface for mass spectroscopy at a sensitivity of detection of a few parts per billion. The sample surface is bombarded by primary energetic particles, such as electrons, ions (e.g., O, Cs), neutrals or even photons, forcing atomic and molecular particles to be ejected from the surface, a process called sputtering. Since some of these sputtered particles carry a charge, a mass spectrometer can be used to measure their mass and charge. Continued sputtering permits measuring of the exposed elements as material is removed. This in turn permits one to construct elemental depth profiles. Although the majority of secondary ionized particles are electrons, it is the secondary ions which are detected and analyzed by the mass spectrometer in this method.

4. LD-MS and LDLPMS

Laser desorption mass spectroscopy (LD-MS) involves the use of a pulsed laser, which induces desorption of sample material from a sample site—effectively, this means vaporization of sample off of the sample substrate. This method is usually only used in conjunction with a mass spectrometer, and can be performed simultaneously with ionization if one uses the right laser radiation wavelength.

When coupled with Time-of-Flight (TOF) measurement, LD-MS is referred to as LDLPMS (Laser Desorption Laser Photoionization Mass Spectroscopy). The LDLPMS method of analysis gives instantaneous volatilization of the sample, and this form of sample fragmentation permits rapid analysis without any wet extraction chemistry. The LDLPMS instrumentation provides a profile of the species present while the retention time is low and the sample size is small. In LDLPMS, an impactor strip is loaded into a vacuum chamber. The pulsed laser is fired upon a certain spot of the sample site, and species present are desorbed and ionized by the laser radiation. This ionization also causes the molecules to break up into smaller fragment-ions. The positive or negative ions made are then accelerated into the flight tube, being detected at the end by a microchannel plate detector. Signal intensity, or peak height, is measured as a function of travel time. The applied voltage and charge of the particular ion determines the kinetic energy, and separation of fragments are due to different size causing different velocity. Each ion mass will thus have a different flight-time to the detector.

One can either form positive ions or negative ions for analysis. Positive ions are made from regular direct photoionization, but negative ion formation requires a higher powered laser and a secondary process to gain electrons. Most of the molecules that come off the sample site are neutrals, and thus can attract electrons based on their electron affinity. The negative ion formation process is less efficient than forming just positive ions. The sample constituents will also affect the outlook of a negative ion spectra.

Other advantages with the LDLPMS method include the possibility of constructing the system to give a quiet baseline of the spectra because one can prevent coevolved neutrals from entering the flight tube by operating the instrument in a linear mode.

5. MALDI-TOF-MS

Since its inception and commercial availability, the versatility of MALDI-TOF-MS has been demonstrated convincingly by its extensive use for qualitative analysis. For example, MALDI-TOF-MS has been employed for the characterization of synthetic polymers (Marie et al., 2000; Wu et al., 1998). peptide and protein analysis (Roepstorff et al., 2000; Nguyen et al., 1995), DNA and oligonucleotide sequencing (Miketova et al., 1997; Faulstich et al., 1997; Bentzley et al., 1996), and the characterization of recombinant proteins (Kanazawa et al., 1999; Villanueva et al., 1999). Recently, applications of MALDI-TOF-MS have been extended to include the direct analysis of biological tissues and single cell organisms with the aim of characterizing endogenous peptide and protein constituents (Li et al., 2000; Lynn et al., 1999; Stoeckli et al., 2001; Caprioli et al., 1997; Chaurand et al., 1999; Jespersen et al., 1999).

The properties that make MALDI-TOF-MS a popular qualitative tool—its ability to analyze molecules across an extensive mass range, high sensitivity, minimal sample preparation and rapid analysis times—also make it a potentially useful quantitative tool. MALDI-TOF-MS also enables non-volatile and thermally labile molecules to be analyzed with relative ease. It is therefore prudent to explore the potential of MALDI-TOF-MS for quantitative analysis in clinical settings. In addition, the application of MALDI-TOF-MS to the detection and/or analysis of blood components is particularly relevant. The ability to quantify intact proteins in biological tissue and fluids presents a particular challenge in the expanding area of proteomics and investigators urgently require methods to accurately measure the absolute quantity of proteins. While there have been reports of quantitative MALDI-TOF-MS applications, there are many problems inherent to the MALDI ionization process that have restricted its widespread use (Kazmaier et al., 1998; Horak et al., 2001; Gobom et al., 2000; Wang et al., 2000; Desiderio et al, 2000). These limitations primarily stem from factors such as the sample/matrix heterogeneity, which are believed to contribute to the large variability in observed signal intensities for analytes, the limited dynamic range due to detector saturation, and difficulties associated with coupling MALDI-TOF-MS to on-line separation techniques such as liquid chromatography. Combined, these factors are thought to compromise the accuracy, precision, and utility with which quantitative determinations can be made.

Because of these difficulties, practical examples of quantitative applications of MALDI-TOF-MS have been limited. Most of the studies to date have focused on the quantification of low mass analytes, in particular, alkaloids or active ingredients in agricultural or food products (Wang et al., 1999; Jiang et al., 2000; Wang et al., 2000; Yang et al., 2000; Wittmann et al., 2001), whereas other studies have demonstrated the potential of MALDI-TOF-MS for the quantification of biologically relevant analytes such as neuropeptides, proteins, antibiotics, or various metabolites in biological tissue or fluid (Muddiman et al., 1996; Nelson et al., 1994; Duncan et al., 1993; Gobom et al., 2000; Wu et al., 1997; Mirgorodskaya et al., 2000). In earlier work it was shown that linear calibration curves could be generated by MALDI-TOF-MS provided that an appropriate internal standard was employed (Duncan et al., 1993). This standard can “correct” for both sample-to-sample and shot-to-shot variability. Stable isotope labeled internal standards (isotopomers) give the best result.

With the marked improvement in resolution available on modern commercial instruments, primarily because of delayed extraction (Bahr et al., 1997; Takach et al., 1997), the opportunity to extend quantitative work to other examples is now possible; not only of low mass analytes, but also biopolymers.

The properties of the matrix material used in the MALDI method are critical. Only a select group of compounds is useful for the selective desorption of proteins and polypeptides. A review of all the matrix materials available for peptides and proteins shows that there are certain characteristics the compounds must share to be analytically useful. Despite its importance, very little is known about what makes a matrix material “successful” for MALDI. The few materials that do work well are used heavily by all MALDI practitioners and new molecules are constantly being evaluated as potential matrix candidates. With a few exceptions, most of the matrix materials used are solid organic acids. Liquid matrices have also been investigated, but are not used routinely.

C. Biological Activity

It is contemplated that analytes of interest may also be assessed for a biological activity. For example, one of skill in the art may choose to assess the biological activity of a blood component when a change in this activity has clinical relevance. Examples of biological activities include, but are not limited to, binding, enzymatically altering, activating, inhibiting, regulating, phosphorylating, utilizing phosphatase activity, ubiquitinating, sumoylating, assisting or inhibiting folding, targeting, transporting, stabilizing, synthesizing, converting, destabilizing, or cleaving.

D. Modifications of Analytes

In another aspect of the invention, analytes may be assessed for a particular modification. For example, it is contemplated that such methods may be particularly useful when modification of an analyte or analytes is indicative of a clinical state. Examples of analyte modifications include, but are not limited to, phosphorylation, ubiquitination, sumoylation, mutation, truncation, glycosylation, acetylation, methylation and hydroxylation. Analyte modifications may also include a change in the folding or assembly of an analyte. Detection and/or analysis of a modification may utilize any suitable immunodetection or mass spectrometry method or methods discussed in previous sections or any method which assays the biological activity of an analyte. Detection and/or analysis of carbohydrate or lipid modifications are also contemplated in accordance with the methods presented in sections V and VI. It will be understood that other methods useful for detecting a modification of an analyte are well known in the art and include, but are not limited to, separation by chromatography, electrophoresis or isoelectric point. Mutational modifications of nucleic acid analytes may be detected by SSCP (single strand conformational polymorphism analysis), DGGE (Denaturing Gradient Gel Electrophoresis), DHPLC (Denaturing High-Performance Liquid Chromatography), CCM (Chemical Cleavage of Mismatches), EMC (Enzyme Mismatch Cleavage), Heteroduplex analysis or DNA microarrays.

E. Nucleic Acid Detection

In alternative embodiments, nucleic acids associated with analytes or nucleic acids as analytes may be detected and/or analyzed using the invention. For example, one of skill in the art may utilize embodiments of the invention to collect and analyze blood peptides or polypeptides based on their association with nucleic acids. Alternatively, it is contemplated that one of skill in the art may utilize embodiments of the present invention to detect nucleic acids when the presence of particular nucleic acids in the bloodstream is indicative of a clinical state. The following is a discussion of nucleic acid detection methods.

Hybridization is defined as the ability of a nucleic acid to selectively form duplex molecules with complementary stretches of DNAs and/or RNAs. Depending on the application envisioned, one would employ varying conditions of hybridization to achieve varying degrees of selectivity of the probe or primers for the target sequence.

Typically, a probe or primer of between 13 and 100 nucleotides, preferably between 17 and 100 nucleotides in length up to 1-2 kilobases or more in length will allow the formation of a duplex molecule that is both stable and selective. Molecules having complementary sequences over contiguous stretches greater than 20 bases in length are generally preferred, to increase stability and selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or mRNA transcripts. It is generally appreciated that conditions can be rendered more stringent by the addition of increasing amounts of formamide.

For certain applications, for example, lower stringency conditions may be used. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In some embodiments, it may be advantageous to employ nucleic acids of defined sequences in combination with an appropriate means, such as a label, for determining hybridization. A wide variety of appropriate indicator means are known in the art, including fluorescent, radioactive, enzymatic or other ligands, such as avidin/biotin, which are capable of being detected. In certain embodiments, one may desire to employ a fluorescent label or an enzyme tag such as urease, alkaline phosphatase or peroxidase, instead of radioactive or other environmentally undesirable reagents. In the case of enzyme tags, calorimetric indicator substrates are known that can be employed to provide a detection means that is visibly or spectrophotometrically detectable, to identify specific hybridization with complementary nucleic acid containing samples.

In general, it is envisioned that probes or primers may be useful as reagents in solution hybridization, as in PCR™, for detection of nucleic acids, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

1. Amplification of Nucleic Acids

Since many nucleic acids are present in relatively low abundance, nucleic acid amplification greatly enhances the ability to detect and/or analyze nucleic acids. The general concept is that nucleic acids can be amplified using paired primers flanking the region of interest. The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids of interest are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemilluminescence, radioactive scintigraphy of incorporated radiolabel or fluorescent label or even via a system using electrical and/or thermal impulse signals.

A number of template-dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al, 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Whereas standard PCR usually uses one pair of primers to amplify a specific sequence, multiplex-PCR (MPCR) uses multiple pairs of primers to amplify many sequences simultaneously (Chamberlan et al., 1990). The presence of many PCR primers in a single tube could cause many problems, such as the increased formation of misprimed PCR products and “primer dimers,” the amplification discrimination of longer DNA fragments and so on. Normally, MPCR buffers contain a Taq Polymerase additive, which decreases the competition among amplicons and the amplification discrimination of longer DNA fragment during MPCR. MPCR products can further be hybridized with sequence-specific probes for verification. Theoretically, one should be able to use as many primers as necessary. However, due to side effects (primer dimers, misprimed PCR products, etc.) caused during MPCR, there is a limit (less than 20) to the number of primers that can be used in a MPCR reaction. See also European Application No. 0 364 255 and Mueller & Wold (1989).

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 discloses a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) discloses a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

2. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products may be visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 1989). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

3. Nucleic Acid Arrays

Microarrays comprise a plurality of polymeric molecules spatially distributed over, and stably associated with, the surface of a substantially planar substrate, e.g., biochips. Microarrays of polynucleotides have been developed and find use in a variety of applications, such as screening and DNA sequencing.

In gene expression analysis with microarrays, an array of “probe” oligonucleotides is contacted with a nucleic acid sample of interest, i.e., target, such as polyA mRNA from a particular tissue type. Contact is carried out under hybridization conditions and unbound nucleic acid is then removed. The resultant pattern of hybridized nucleic acid provides information regarding the genetic profile of the sample tested. Methodologies of gene expression analysis on microarrays are capable of providing both qualitative and quantitative information.

A variety of different arrays which may be used are known in the art. The probe molecules of the arrays which are capable of sequence specific hybridization with target nucleic acid may be polynucleotides or hybridizing analogues or mimetics thereof, including: nucleic acids in which the phosphodiester linkage has been replaced with a substitute linkage, such as phophorothioate, methylimino, methylphosphonate, phosphoramidate, guanidine and the like; nucleic acids in which the ribose subunit has been substituted, e.g., hexose phosphodiester; peptide nucleic acids; and the like. The length of the probes will generally range from 10 to 1000 nts, where in some embodiments the probes will be oligonucleotides and usually range from 15 to 150 nts and more usually from 15 to 100 nts in length, and in other embodiments the probes will be longer, usually ranging in length from 150 to 1000 nts, where the polynucleotide probes may be single- or double-stranded, usually single-stranded, and may be PCR fragments amplified from cDNA.

The probe molecules on the surface of the substrates will correspond to selected nucleic acid sequences being analyzed and be positioned on the array at a known location so that positive hybridization events may be correlated to the presence of a particular nucleic acid sequence in the physiological source from which the target nucleic acid sample is derived. The substrates with which the probe molecules are stably associated may be fabricated from a variety of materials, including plastics, ceramics, metals, gels, membranes, glasses, and the like. The arrays may be produced according to any convenient methodology, such as preforming the probes and then stably associating them with the surface of the support or growing the probes directly on the support. A number of different array configurations and methods for their production are known to those of skill in the art and disclosed in U.S. Pat. Nos. 5,445,934, 5,532,128, 5,556,752, 5,242,974, 5,384,261, 5,405,783, 5,412,087, 5,424,186, 5,429,807, 5,436,327, 5,472,672, 5,527,681, 5,529,756, 5,545,531, 5,554,501, 5,561,071, 5,571,639, 5,593,839, 5,599,695, 5,624,711, 5,658,734, 5,700,637, and 6,004,755.

Following hybridization, where non-hybridized labeled nucleic acid is capable of emitting a signal during the detection step, a washing step is employed where unhybridized labeled nucleic acid is removed from the support surface, generating a pattern of hybridized nucleic acid on the substrate surface. A variety of wash solutions and protocols for their use are known to those of skill in the art and may be used.

Where the label on the target nucleic acid is not directly detectable, one then contacts the array, now comprising bound target, with the other member(s) of the signal-producing system that is being employed. For example, where the label on the target is biotin, one then contacts the array with streptavidin-fluorescer conjugate under conditions sufficient for binding between the specific binding member pairs to occur. Following contact, any unbound members of the signal-producing system will then be removed, e.g., by washing. The specific wash conditions employed will necessarily depend on the specific nature of the signal producing system that is employed, and will be known to those of skill in the art familiar with the particular signal-producing system employed.

The resultant hybridization pattern(s) of labeled nucleic acids may be visualized or detected in a variety of ways, with the particular manner of detection being chosen based on the particular label of the nucleic acid, where representative detection means include scintillation counting, autoradiography, fluorescence measurement, calorimetric measurement, light emission measurement and the like.

Prior to detection or visualization, where one desires to reduce the potential for a mismatch hybridization event to generate a false positive signal on the pattern, the array of hybridized target/probe complexes may be treated with an endonuclease under conditions sufficient such that the endonuclease degrades single stranded, but not double stranded DNA. A variety of different endonucleases are known and may be used, where such nucleases include: mung bean nuclease, SI nuclease, and the like. Where such treatment is employed in an assay in which the target nucleic acids are not labeled with a directly detectable label, e.g., in an assay with biotinylated target nucleic acids, the endonuclease treatment will generally be performed prior to contact of the array with the other member(s) of the signal producing system, e.g., fluorescent-streptavidin conjugate. Endonuclease treatment, as described above, ensures that only end-labeled target/probe complexes having a substantially complete hybridization at the 3′ end of the probe are detected in the hybridization pattern.

Following hybridization and any washing step(s) and/or subsequent treatments, as described above, the resultant hybridization pattern is detected. In detecting or visualizing the hybridization pattern, the intensity or signal value of the label will be not only be detected but quantified, by which is meant that the signal from each spot of the hybridization will be measured and compared to a unit value corresponding the signal emitted by known number of end-labeled target nucleic acids to obtain a count or absolute value of the copy number of each end-labeled target that is hybridized to a particular spot on the array in the hybridization pattern.

F. Detection of Carbohydrates

It is contemplated that the invention may be utilized to detect and analyze carbohydrates present in body fluids. For example, many biologically interesting proteins are glycosylated at their asparagine, serine, and threonine residues. In fact, glycosylation is now recognized as being more ubiquitous and structurally varied than all other types of posttranslational modifications combined. In some instances, changes in glycosylation may be indicative of a disease state (see Dube et al., 2005). Therefore, for example, one of skill in the art may utilize embodiments of the invention to detect and/or analyze carbohydrates in order to assay the glycosylation state of an analyte.

Modern mass spectrometry, featuring most prominently the matrix-assisted laser desorption/ionization (MALDI) and electrospray ionization (ESI) methodologies, has been employed for the structural elucidation of carbohydrates (Mechref et al., 2002; Dell et al., 2001). Any suitable mass spectrometry or immunodetection method described above may also be employed for the detection and/or analysis of carbohydrates or carbohydrate-containing analytes. Other methods which may be used to analyze carbohydrates are hydrophilic interaction and adsorption chromatography, liquid chromatography including reversed-phase HPLC, anion-exchange chromatography, and lectin affinity column chromatography (Mechref et al., 2002). Still other methods for detecting and/or analyzing carbohydrates are known in the art and are contemplated as applicable to the present invention. For example, U.S. Pat. Nos. 5,308,460 and 6,858,135 disclose methods for detection and analysis of carbohydrates and are incorporated herein by reference. U.S. Pat. No. 5,031,449 discloses the use of electrodes as applicable to carbohydrate analysis and is also incorporated herein by reference. WO9845469A1 and U.S. Pat. No. 5,472,582 disclose fluorometric assays for carbohydrate analysis, and these disclosures are incorporated herein by reference. U.S. Pat. No. 6,844,166 discloses carbohydrate-binding ligands and their use in detection and analysis of carbohydrates and is incorporated herein by reference.

Additionally, carbohydrate detection is uniquely useful for diagnosis and monitoring of diseases in which blood sugars are misregulated, such as diabetes. U.S. Patent Application No. 20050038329A1 and U.S. Pat. No. 5,972,631 disclose assays for glucose and sucrose and are incorporated herein by reference.

In another aspect of the invention, detection and/or analysis of carbohydrates may aid in the diagnosis or monitoring of a bacterial infection. For example, Gram-negative bacteria are known to have in common the possession of at least one lipo-polysaccharide or other lipo-polycarbohydrate antigen, while Gram-positive bacteria are known to possess the common characteristic of having at least one carbohydrate antigen that is a lipo-teichoic acid or teichoic acid or a derivative of either. It is contemplated that the methods described above for detection and/or analysis of carbohydrates are applicable to the diagnosis and/or monitoring of a bacterial infection.

G. Detection of Lipids

It is contemplated that the invention may be useful in detecting and/or analyzing lipids present in body fluids as a means of diagnosing and/or monitoring a clinical state. It will be understood that methods for the detection of lipids are known in the art and contemplated as methods for detecting and/or analyzing lipids in the context of the invention. For example, methods for using lipid recognition proteins and employing lipid phosphatase assays and fluorescence-based assays which are applicable to lipid detection are described in U.S. Patent Application No. 20040096923A1 and No. 20030100028A1, and these disclosures are incorporated herein by reference. U.S. Pat. No. 5,470,714 discloses methods for analysis of fatty acids and is also incorporated herein by reference. U.S. Patent Application No. 20040259187A1 discloses methods for measuring lipid antioxidant activity that can be used for diagnosing and protecting against disorders that arise from excess free radicals present in a subject and are incorporated herein by reference. In addition, U.S. Pat. No. 5,187,068 describes methods for detecting and measuring lipoproteins and for determining the corresponding lipid and apolipoprotein components and is incorporated herein by reference.

### IV. In Vivo Administration

Clinical applications of the present invention contemplate the use of the methodology and/or devices described herein for in vivo applications, particularly diagnostic applications for disease. In addition to lack of toxicity, in vivo uses would also benefit from various pharmaceutical additives, such as coatings with antibacterial and antifungal agents, delaying agents, anti-inflammatories, or dissolving/soluble markers. In particular, the device and agents should be sterile, and thus the device may be stored in sterile, pharmaceutically acceptable buffers.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### V. References

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 4,683,195
U.S. Pat. No. 4,683,202
U.S. Pat. No. 4,800,159
U.S. Pat. No. 4,883,750
U.S. Pat. No. 4,952,500
U.S. Pat. No. 5,031,449
U.S. Pat. No. 5,187,068
U.S. Pat. No. 5,242,974
U.S. Pat. No. 5,279,721
U.S. Pat. No. 5,308,460
U.S. Pat. No. 5,384,261
U.S. Pat. No. 5,405,783
U.S. Pat. No. 5,412,087
U.S. Pat. No. 5,424,186
U.S. Pat. No. 5,429,807
U.S. Pat. No. 5,436,327
U.S. Pat. No. 5,445,934
U.S. Pat. No. 5,464,765
U.S. Pat. No. 5,470,714
U.S. Pat. No. 5,472,582
U.S. Pat. No. 5,472,672
U.S. Pat. No. 5,527,681
U.S. Pat. No. 5,529,756
U.S. Pat. No. 5,532,128
U.S. Pat. No. 5,545,531
U.S. Pat. No. 5,554,501
U.S. Pat. No. 5,556,752
U.S. Pat. No. 5,561,071
U.S. Pat. No. 5,563,055
U.S. Pat. No. 5,571,639
U.S. Pat. No. 5,593,839
U.S. Pat. No. 5,599,695
U.S. Pat. No. 5,624,711
U.S. Pat. No. 5,658,734
U.S. Pat. No. 5,700,637
U.S. Pat. No. 5,757,994
U.S. Pat. No. 5,788,166
U.S. Pat. No. 5,789,215
U.S. Pat. No. 5,838,002
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,849,481
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,336
U.S. Pat. No. 5,882,864
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,925,565
U.S. Pat. No. 5,928,862
U.S. Pat. No. 5,928,869
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,819
U.S. Pat. No. 5,935,825
U.S. Pat. No. 5,939,291
U.S. Pat. No. 5,942,391
U.S. Pat. No. 5,945,100
U.S. Pat. No. 5,972,631
U.S. Pat. No. 5,981,274
U.S. Pat. No. 5,986,258
U.S. Pat. No. 5,994,136
U.S. Pat. No. 5,994,624
U.S. Pat. No. 6,004,755
U.S. Pat. No. 6,013,516
U.S. Pat. No. 6,702,790
U.S. Pat. No. 6,844,166
U.S. Pat. No. 6,858,135
U.S. Pat. No. RE 35,413
U.S. Patent Appln. 20020098595
U.S. Patent Appln. 20030100028
U.S. Patent Appln. 20040096923
U.S. Patent Appln. 20040259187
U.S. Patent Appln. 20050038329
Bahr et al., J. Mass Spectrom., 32:1111-1116, 1997.
Bentzley et al, Anal Chem., 68(13):2141-2146, 1996.
Bucknall et al., J. Am. Soc. Mass Spectrom., 13(9):1015-1027, 2002.
Caprioli et al., Anal. Chem., 69:4751, 1997.
Chamberlan et al., In: PCR Protocols, eds. Innis, Gelfand, Sninsky, White (Academic Press, NY, 272-281, 1990.
Chaurand et al, Anal Chem., 71(23):5263-5270, 1999.
Chen et al., Nat. Biotechnol., 19:537-542, 2001.
De Jager et al., Semin Nucl Med 23(2):165-179, 1993.
Dell et al., Science, 291:2351-2356, 2001.
Desiderio et al., J. Mass Spectrom., 35(6):725-733, 2000.
Desiderio et al., Methods Mol. Biol., 61:57-65, 1996.
Doolittle et al., Methods Mol Biol., 109:215-237, 1999.
Dube and Bertozzi, Nat Rev Drug Discov., 4(6):477-88, 2005.
Duncan et al., Rapid Commun. Mass Spectrom., 7(12):1090-1094, 1993.
European Application No. 0 364 255
European Application No. 320 308
European Application No. GB 2 202 328
European Application No. 329 822
Faulstich et al., Anal. Chem., 69(21):4349-4353, 1997.
Fenn et al., Science, 246(4926):64-71, 1989.
Frohman, In: PCR Protocols: A Guide To Methods And Applications, Academic Press, N.Y., 1990.
Gobom et al., Anal. Chem., 72(14):3320-3326, 2000.
Gulbis and Galand, Hum Pathol 24(12):1271-1285, 1993.
Horak et al., Rapid Commun. Mass Spectrom., 15(4):241-248, 2001.
Innis et al, Proc. Natl. Acad. Sci. USA, 85(24):9436-9440, 1988.
Jespersen et al., Anal Chem., 71(3):660-666, 1999.
Jiang et al., J. Agric. Food Chem., 48:3305, 2000.
Kabarle et al., Anal. Chem. 65(20):972A-986A, 1993.
Kanazawa et al., Biol. Pharm. Bull., 22(4):339-346, 1999.
Kazmaier et al., Anesthesiology, 89(4):831-817, 1998.
Kern et al., Blood, 103(2):679-688, 2004.
Kiernan et al., Anal. Biochem., 301(1):49-56, 2002a.
Kiernan et al., Biochem. Biophys. Res. Commun., 292(2):401-5, 2002b.
Kwoh et al., Proc. Nat. Acad. Sci. USA, 86: 1173, 1989.
Li et al., Trends Biotechnol., 18:151, 2000.
Lovelace et al., J. Chromatogr., 562(1-2):573-584, 1991.
Lynn et al., J. Mol. Evol., 48(5):605-614, 1999.
Marie et al., Anal. Chem., 72(20):5106-5114, 2000.
Mechref et al., Chem. Rev., 102:321-370, 2002.
Miketova et al., Mol. Biotechnol., 8(3):249-253, 1997.
Mirgorodskaya et al., Rapid Commun. Mass Spectrom., 14(14):1226-1232, 2000.
Muddiman et al., Fres. J. Anal. Chem., 354:103, 1996.
Mueller and Wold, Science 246, 780-786, 1989.
Nakamura et al., In: Handbook of Experimental Immunology (4^{th} Ed.), Weir et al., (eds). 1:27, Blackwell Scientific Publ., Oxford, 1987.
Nedelkov et al., J. Mol. Recognit., 16(1):15-9, 2003.
Nedelkov et al., Proteomics, 1(11): 1441-6, 2001.
Nelson et al., Anal. Chem., 66:1408, 1994.
Nguyen et al., J. Chromatogr. A., 705(1):21-45, 1995.
Ohara et al., Proc. Nat'l Acad. Sci. USA, 86: 5673-5677, 1989.
PCT Application PCT/US87/00880
PCT Application PCT/US89/01025
PCT Application WO 88/10315
PCT Application WO 90/07641
PCT Application WO89/06700
PCT Appln. WO 98/45469A1
Roepstorff, EXS., 88:81-97, 2000.
Sambrook et al, In: Molecular Cloning: A Laboratory Manual, 2nd Ed., Cold Spring Harbor Press, Cold Spring Harbor, N.Y., 1989.
Stoeckli et al., Nat. Med., 7(4):493-496, 2001.
Takach et al., J. Protein Chem., 16:363, 1997.
Villanueva et al., Enzyme Microb. Technol., 29:99, 1999.
Walker et al., Proc. Nat'l Acad. Sci. USA, 89:392-396 1992.
Wang et al., Anal. Chem., 72(21):5285-5289, 2000.
Wang et al., J. Agric. Food. Chem., 47:1549, 1999.
Wang et al., J. Agric. Food. Chem., 47:2009, 1999.
Wittmann et al., Biotechnol. Bioeng., 72:642, 2001.
Wu et al., Anal. Chem., 70:456 A, 1998.
Wu et al., Biochem. Biophys. Res. Commun., 233(1):221-226, 1997.
Wu et al., Biochim. Biophys. Acta, 1466:315-327, 2000.
Yang et al., J. Agric. Food. Chem., 48:3990, 2000.
Zhong et al, Clin. Chem. ACTA., 313:147, 2001.
Zweigenbaum et al., Anal. Chem., 71(13):2294-300, 1999.
Zweigenbaum et al., J. Pharm. Biomed. Anal., 23(4):723-733, 2000.

1. A device for insertion into a subject, the device comprising:
(a) a housing suitable for disposition in a subject's body fluid;
(b) a probe comprising a support coupled to the housing; and
(c) a binding agent located on or in the support.

2. The device of claim 1, wherein the housing is a hypodermic needle or syringe.

3. The device of claim 1, wherein the housing is a catheter.

4. The device of claim 1, wherein the support is a stent.

5. The device of claim 1, wherein the support is comprised of a polymer or resin.

6. The device of claim 1, wherein the support is a filter.

7. The device of claim 1, wherein the support is comprised of glass.

8. The device of claim 1, wherein the support is a chip.

9. The device of claim 1, wherein the support is porous.

10. The device of claim 1, wherein the support is a membrane.

11. The device of claim 1, wherein the binding agent is a peptide.

12. The device of claim 1, wherein the binding agent is a polypeptide.

13. The device of claim 12, wherein the polypeptide is an antibody or a receptor.

14. The device of claim 1, wherein the binding agent is a drug nucleic acid, glycolipid, carbohydrate, toxin, hapten, diagnostic agent, or enzyme substrate.

15-21. (canceled)

22. The device of claim 1, further comprising a means for positioning the probe inside or outside of the housing.

23. The device of claim 22, wherein the means is a cable, plunger or piston.

24. The device of claim 22, wherein the housing is a hypodermic needle or syringe.

25. The device of claim 22, wherein the housing is a catheter.

26. The device of claim 1, wherein the housing and/or support is coated with an anticoagulant or blocking agent.

27. The device of claim 1, wherein the body fluid is selected from blood, serum, sputum, saliva, urine, cerebrospinal fluid, or ascites.

28. A method of recovering an analyte comprising:
(i) providing a device for insertion into a fluid located in a subject, the device comprising:
(a) a housing;
(b) a probe comprising a support coupled to the housing; and
(c) a binding agent located on or in the support,
(ii) inserting the device into the fluid of the subject such that the support is brought into contact with the subject's fluid; and
(iii) removing the device and/or support from the subject.

29-36. (canceled)

37. The method of claim 28, further comprising purifying the analyte away from other molecules bound to the support.

38. The method of claim 37, wherein purifying comprises removing the analyte from support.

39. The method of claim 38, further comprising centrifugation, filtration, enzymatic digestion or immunologic separation of the analyte removed from the support.

40. The method of claim 37, further comprising purifying the analyte by removing other molecules bound to the support.

41. The method of claim 28, wherein the bloodstream is an arterial bloodstream.

42. The method of claim 28, wherein the blood stream is a venous bloodstream.

43. The method of claim 28, wherein the device is left in the subject's blood stream for between one minute and 24 hours.

44. The method of claim 43, wherein the device is left in the subject's blood stream for about two minutes.

45. The method of claim 43, wherein the device is left in the subject's blood stream for about an hour.

46. The method of claim 43, wherein the device is left in the subject's blood stream for about six hours.

47. The method of claim 28, wherein binding of the analyte to the support results in a detectable event.

48. The method of claim 47, wherein the detectable event comprises a color change, a fluorescent signal being generated, a chemical reaction, or generation of an electrical signal.

49. The method of claim 28, wherein the device further delivers an exogenous agent to the subject.

50. The method of claim 28, wherein the body fluid is selected from blood, serum, sputum, saliva, urine, cerebrospinal fluid, or ascites.

51. A method of assaying for a body fluid analyte comprising:
(i) providing a device for insertion into a subject's body fluid, the device comprising:
(a) a housing suitable for disposition in a subject's body fluid;
(b) a probe comprising a support coupled to the housing; and
(c) a binding agent coupled to the probe with at least a support,
(ii) inserting the device into the body fluid of the subject such that the support is brought into contact with the subject's body fluid;
(iii) removing the device and/or support from the subject;
(iv) analyzing the support for the presence, amount and/or state of the analyte.

52. The method of claim 51, further comprising removing the analyte from the support prior to step (iv).

53. The method of claim 51, wherein analyzing comprises mass spectrometry.

54. The method of claim 53, wherein the mass spectrometry is secondary ion mass spectrometry, laser desorption mass spectrometry, matrix assisted laser desorption mass spectrometry, or electrospray mass spectrometry.

55. The method of claim 51, wherein analyzing comprises separation of the analyte by size charge, or affinity.

56. The method of claim 51, wherein analyzing comprises assessing immunologic reactivity.

57. The method of claim 51, wherein analyzing comprises assessing biological activity.

58. The method of claim 51, wherein analyzing the state of an analyte comprises detecting a modification of the analyte.

59. The method of claim 58, wherein the modification is phosphorylation, ubiquitination, cleavage, sulfation, glycosylation, mutation or truncation.

60. The method of claim 58, wherein the modification comprises a change in folding or assembly.

61. The device of claim 1, further comprising that the probe is suitable for direct insertion into a mass spectrometer and subsequent analysis.

62. The method of claim 51, wherein the body fluid is selected from blood, serum, sputum, saliva, urine, cerebrospinal fluid, or ascites.

63. A device suitable for insertion into a subject's body fluid, the device comprising:
(a) a probe comprising a support coupled to the housing; and
(b) a binding agent located on or in the support.

64-68. (canceled)

69. A device suitable for insertion into a subject's body fluid, the device comprising:
(a) a housing having a portion sized for placement in a subject's blood vessel;
(b) a probe comprising a support coupled to the housing; and
(c) a binding agent located on or in the support.

70-77. (canceled)

78. A device suitable for insertion into a subject's body fluid, the device comprising:
(a) a housing having a portion sized for placement in a subject's blood vessel;
(b) a probe having a distal portion sized for insertion through the housing; and
(c) a binding agent coupled to the probe with at least a support.

79-86. (canceled)

87. A device suitable for insertion into a subject's body fluid, the device comprising: a probe; and a binding agent coupled to the probe with at least a support; the binding agent exhibiting affinity for one or more components found in said body fluid.

88-93. (canceled)
